(19) United States
(12) Patent Application Publication
(10) Pub. No.: US 2025/0275916 A1
(43) Pub. Date: Sep. 4, 2025

(54) MEDICATED EYELID CLEANSING COMPOSITIONS

(71) Applicant: OCuSOFT, Inc., Rosenberg, TX (US)

(72) Inventors: Paula Hooper, Rosenberg, TX (US); Paramita Sarkar, Rosenberg, TX (US)

(21) Appl. No.: 19/069,051

(22) Filed: Mar. 3, 2025

Related U.S. Application Data
(60) Provisional application No. 63/561,066, filed on Mar. 4, 2024.

(51) Int. Cl. A61K 9/00; A61K 31/196; A61K 31/4172; A61K 31/573; A61K 31/7048; A61K 47/26

(52) U.S. Cl. CPC A61K 9/0048; A61K 31/196; A61K 31/4172; A61K 31/573; A61K 31/7048; A61K 47/26

(57) ABSTRACT

A medicated eyelid cleansing composition comprising a medicated scrub composition and an eyelid cleansing composition. The medicated scrub composition has one or more antibacterial and anti-inflammatory agents, including azithromycin and diclofenac sodium or dexamethasone phosphate. The medicated scrub composition can further include a combination of metronidazole and ivermectin. The composition is optimized for physiological compatibility and can be combined with a suitable substrate for use as a medicated eyelid cleanser.

### CROSS-REFERENCE TO RELATED APPLICATION

This application claims the benefit of Provisional U.S. Patent Application No. 63/561,066, filed Mar. 4, 2024, entitled “Medicated Eyelid Scrub Composition”, the entire content and disclosure of which, both express and implied, is incorporated herein by reference.

### TECHNICAL FIELD

The present invention pertains to a medicated eyelid cleansing composition designed to possess an enhanced therapeutic effect and specifically formulated for use in the ocular region. In particular, the medicated eyelid cleansing composition includes a medicated eyelid scrub composition having a therapeutically effective amount of one or more active pharmaceutical ingredients.

### BACKGROUND

The eyelids play a crucial role in maintaining overall ocular health by shielding the eyes from external threats such as airborne contaminants or approaching objects. They contain several glands, including the lacrimal and meibomian glands, which produce a vital layer of tear film that keeps the eyes moist and healthy. Every time an individual blinks, the eyelids create a fresh tear film that lubricates the eye's surface and clears out foreign bodies.

However, the eyelids are susceptible to various issues that can cause discomfort, particularly for contact lens wearers, and can lead to severe complications. One such issue is blepharitis, a common chronic inflammation characterized by scaly crust on the lid margins. Blepharitis can be caused by a bacterial infection, allergies, or seborrhea of the face and scalp. Regular cleaning of the eyelids is essential to remove excess oil, debris, and dead skin that can exacerbate the problem.

Blepharitis can also lead to other complications such as an internal hordeolum, a bacterial infection of the skin at the edge of the lid, or an external hordeolum, commonly known as a stye, which is an infection of the meibomian glands. Styes are red, tender bumps that typically develop fully within three days and cause pain, redness, and tenderness of the lid margins. Regular cleaning of the eyelid margins can help minimize such conditions. Another issue is a chalazion, an inflammation of the meibomian glands inside the eyelid, which may require surgical intervention if left untreated.

Proper eyelid hygiene can minimize the severity of these problems, including rosacea and seborrhea. Cleaning the eyelashes, eyelids, or the periocular area, and can serve to reduce the presence of harmful bacteria that can cause infections or inflammation in patients.

However, ordinary skin cleansers are often not suitable for use near the eye and may be harsh on the thin skin in the ocular area. Typical skin cleanser compositions employ strong detergents and have a pH far outside a comfortable range. Existing skin ointments are also often unsuitable for managing eyelid health due to their thick and heavy texture. They can be challenging to use around the eyes without getting into the eyes and may not permit flexible use during the day. These factors can lead to a lack of compliance with the recommended application frequency, hindering the effectiveness of the treatment.

To manage eyelid health effectively, an ideal eyelid scrub composition should be gentle enough for sensitive skin around the eyes and contain a therapeutic amount of antibacterial and anti-inflammatory medication to help manage eyelid conditions such as blepharitis. Regular cleansing of the eyelids with this type of composition can help prevent and manage eyelid conditions, thereby maintaining healthy eyes.

Traditional lid scrubs have been widely used for mechanical cleansing and debris removal. However, additional therapeutic benefits are needed for patients experiencing bacterial overgrowth and associated inflammation.

### SUMMARY

One or more embodiments of the invention disclose a medicated eyelid cleansing composition for addressing both infection and inflammation simultaneously of the eyelids. The medicated eyelid cleansing composition of the present invention comprises: (A) a medicated scrub composition, and (B) an eyelid cleansing composition. The medicated scrub composition has therapeutically effective amounts of one or more antibacterial and anti-inflammatory agents, including azithromycin and diclofenac sodium or dexamethasone phosphate. The medicated scrub composition can further include a combination of metronidazole and ivermectin. The composition is optimized for physiological compatibility and can be combined with a suitable substrate for use as a medicated eyelid cleanser. The pH of the medicated eyelid cleansing composition is adjusted to around 6 to 7.5. The medicated eyelid cleansing composition can be foaming or non-foaming formulation and it can be in the form of a liquid, cream, lotion, paste, spray, mist, gel or ointment.

In one embodiment, the medicated eyelid scrub composition has: (i) azithromycin; (ii) sodium chloride solution; (iii) phenyl ethyl alcohol; and (iv) polysorbate 80. In one embodiment, the medicated eyelid scrub composition further includes diclofenac sodium. In another embodiment, the medicated eyelid scrub composition further includes dexamethasone phosphate. The medicated eyelid scrub composition is mixed with a first eyelid cleansing composition. The medicated eyelid scrub composition is around 6%-10% w/w and the first eyelid cleansing composition is around 90%-94% w/w of the medicated eyelid cleansing composition.

According to an embodiment, the medicated eyelid scrub composition further comprises: about 0.1%-2.0% w/w azithromycin dihydrate; about 0.5%-5% w/w of 0.9% sodium chloride solution; about 0.001%-0.05% w/w phenylethyl alcohol; and about 1%-5% polysorbate 80.

In one specific embodiment, the medicated eyelid scrub composition further comprises: about 0.1%-2.0% w/w azithromycin dihydrate; about 0.05%-4.0% w/w diclofenac sodium; about 0.5%-5% w/w of 0.9% sodium chloride solution; about 0.001%-0.05% w/w phenylethyl alcohol; and about 1%-5% polysorbate 80.

In another specific embodiment, the medicated eyelid scrub composition further comprises: about 0.1%-2.0% w/w azithromycin dihydrate; about 0.05%-4.0% w/w dexamethasone phosphate; about 0.5%-5% w/w of 0.9% sodium chloride solution; about 0.001%-0.05% w/w phenylethyl alcohol; and about 1%-5% polysorbate 80.

The first eyelid cleansing composition can include Cocoamphodiacetate Disodium, D-Panthenol, Decyl Polygluoside, Methyl Gluceth-20, Modified Ringer's Solution, PEG-120 Methyl Glucose Dioleate, POE-80 Sorbitan Monolaurate, Polyaminopropyl Biguanide, 1,2-Hexanediol and 1,2-Octanediol.

In another embodiment, the medicated eyelid scrub composition has metronidazole and ivermectin. The medicated eyelid scrub composition is mixed with a second eyelid cleansing composition and a plasticized anhydrous ointment base consisting of mineral oil and polyethylene. The second eyelid cleansing composition further comprises: cocoamphodiacetate disodium, D-Panthenol, Decyl Glucoside, Methyl Gluceth-20, Modified Ringer's Solution, PEG-120 Methyl Glucose Dioleate, PEG-80, Sorbitan Monolaurate, polyaminopropyl biguanide, 1,2-Hexanediol, Caprylyl Glycol, Polysorbate 20, Melaleuca Alternifolia (Tea Tree) Leaf Oil, Cocos Nucifera (Coconut) Oil. The medicated eyelid scrub composition is around 6%-10% w/w, the second eyelid cleansing composition is around 20%-50% w/w, and the ointment base is around 20%-50% w/w of the medicated eyelid cleansing composition

### DETAILED DESCRIPTION

The term and phrases “invention,” “present invention,” “instant invention,” and similar terms and phrases as used herein are non-limiting and are not intended to limit the present subject matter to any single embodiment, but rather encompass all possible embodiments as described.

As used herein, all weight percentages (wt. %) are based on the total wt. % of the skin care composition, unless otherwise specified. Additionally, all composition percentages are based on totals equal to 100 wt. %, unless otherwise specified.

The compositions and methods for their use can “comprise,” “consist essentially of,” or “consist of” any of the ingredients or steps disclosed throughout the specification and claims. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients of the present invention and do not exclude other ingredients or elements described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

As used herein, the term “therapeutically effective amount” of a composition or active ingredient refers to an amount sufficient to elicit the desired biological response. The terms composition and active ingredient are used interchangeably herein. As will be appreciated by those of ordinary skill in this art, and effective amount of a substance may vary depending on such factors as the desired biological endpoint, the patient, etc. The terms effective amount and therapeutically effective amount may be used interchangeably herein.

Unless otherwise indicated, percentages or concentrations refer to percentages or concentrations by weight (ie, % (w/w)). Unless otherwise specified, all ranges are inclusive, for example, “0.5 to 5” includes the 0.5 and 5 endpoints.

The present invention discloses a medicated eyelid scrub composition comprising a therapeutically effective amount of one or more active ingredients. In one exemplary embodiment, the medicated eyelid scrub composition comprises a mixture of an antibiotic and anti-inflammatory agents.

The medicated eyelid scrub composition can further include one or more antimicrobial and preservative agents, antibacterial agents, nonsteroidal anti-inflammatory agents, polyols, surfactants, stabilizers, osmolality adjusters, moisturizers, and purified water. The medicated eyelid scrub is mixed with an eyelid cleansing composition to form a medicated eyelid cleansing composition which is configured to be gentle enough for use in the ocular area and effective in the treatment of associated symptoms and conditions.

One or more embodiments of the eyelid cleansing composition comprise antimicrobial and preservative agents. Exemplary preservative agents with significant activity against microbes are polyaminopropyl biguanide and phenylethyl alcohol. Polyaminopropyl biguanide is also named polyhexamethylene biguanide and polyhexamethylene biguanide hydrochloride. These agents are ideal antimicrobials for the ocular area as they are compatible with a wide range of topical composition excipients, surfactant systems, and vehicles. Antimicrobial and preservative agents are present in the composition up to 5% wt/wt.

Embodiments of the eyelid cleansing composition comprise a blend of one or more surfactants that are optimized for foaming capability, cleansing, and delivery of active ingredients to the surface of the skin. Suitable surfactants can include amphoteric surfactants, anionic surfactants, and nonionic surfactants. Suitable amphoteric surfactants include, but are not limited to alkyldimethyl betaines, alkylamido betaines, sulfobetaines, and imidazoline amphoterics. Suitable anionic surfactants include, but are not limited to fatty alcohol sulfates, alpha olein sulfonates, sulfosuccinates, sarcosinates, phosphate esters, and carboxylates. Suitable nonionic surfactants include, but are not limited to alkanolamides, ethoxylated amides, esters, alkylated alcohols, alkylpolyglucosides, amine oxides, sorbitan esters, and ethoxylates. A preferred surfactant system of the composition comprises cocoamphodiacetate disodium, polyoxyethylene 80 sorbitan monolaurate, decyl polyglucoside, and polysorbate 80. Each surfactant is present in the composition up to 25% wt/wt.

The eyelid cleansing composition further includes one or more polyols having 2-12 carbon atoms. Preferably, at least one 1,2-diol compound is included. The 1,2-diol can be included in the composition for its antimicrobial enhancing activity. Preferred 1,2-diols are selected from the group consisting of 1,2-hexanediol, 1,2-octanediol, 1,2-decanediol, and mixtures thereof. 1,2-hexanediol is a synthetic preservative with a broad spectrum anti-microbial activity. Similarly, 1,2-octanediol is another active substance which functions as a preservative and antimicrobial. Polyols are each present in composition in the range of 0.01 to 5% wt/wt.

In another embodiment of this invention, the eyelid cleansing composition comprises one or more moisturizers, which are chemicals that prevent the loss of water through the skin. These moisturizers work by creating a protective film over the skin, preventing water from evaporating, or by attracting moisture from the air into the skin through hydroscopic molecules. Suitable moisturizers include, but are not limited to, methyl gluceth-20, sorbitol, glycerine, propylene glycol, carboxylates, amino acids, glucoside derivatives, urea, lactates, and pantothenic acid derivatives such as panthenol, D-panthenol, and D, L-panthenol. Moisturizers in the composition are each in the range of 0.05 to 20% wt/wt.

The eyelid cleansing composition additionally includes a stabilizer that prolongs the life of the foam by reducing the surface tension of the composition. A foam stabilizer is a surfactant that has both hydrophilic and hydrophobic properties. The hydrophobic portion of the foam stabilizer interacts with the air at the surface of the composition, while the hydrophilic part interacts with the aqueous portion. An exemplary example of a foam stabilizer is PEG-120 methyl glucose dioleate. A suitable range for a foam stabilizer in the composition is 0.1 to 5% wt/wt.

According to an embodiment, the eyelid cleansing composition comprises a modified Ringer's solution. An isotonic aqueous solution of electrolytes that is physiologically compatible with human tissue is called a modified Ringer's solution. This solution typically comprises sodium chloride, potassium chloride, calcium chloride, and water. When incorporated with surfactants in a composition, a modified Ringer's solution helps to prevent the removal of water from human tissue. Preferably, the modified Ringer's solution comprises 0.05 to 1.2 wt. % sodium chloride, 0.005 to 0.5 wt. % potassium chloride, 0.005 to.5 wt. % calcium chloride, and purified water.

In an exemplary embodiment of the invention, the medicated scrub composition comprises an antibiotic agent. Antibiotics are a class of drugs that are used to treat bacterial infections. They work by killing or inhibiting the growth of bacteria that cause infections. Antibiotics can be classified into different types, such as penicillins, cephalosporins, tetracyclines, macrolides, and fluoroquinolones, among others. They can be taken orally, applied topically, or injected into the body, depending on the type of infection and the severity of the illness. Suitable antibiotics include, but are not limited to, azithromycin, mupirocin, erythromycin, and salts thereof. For instance, in one embodiment, azithromycin dihydrate is used as the antibiotic. A therapeutically effective amount of antibiotic depends on the specific agent, but is preferably between 0.5 and 3% wt/wt.

In yet another embodiment, the medicated scrub composition comprises an anti-inflammatory agent. Anti-inflammatory agents are medications used to reduce inflammation in the body. Inflammation is a natural response by the immune system to injury, infection, or irritation, but it can also cause pain, swelling, and other symptoms. Anti-inflammatory drugs work by blocking or reducing the production of certain chemicals in the body that cause inflammation. Some examples of anti-inflammatory agents include nonsteroidal anti-inflammatory drugs (NSAIDs), corticosteroids, and immunosuppressants. Suitable anti-inflammatory agents include, but are not limited to, diclofenac, ketorolac, bromfenac, and salts thereof. For instance, in one embodiment diclofenac sodium is used as the anti-inflammatory agent. Preferably, the anti-inflammatory agent is an NSAID and is present in the range of 0.1 to 5% wt/wt.

According to an embodiment, the medicated eyelid cleansing composition is free of traditional pH modifiers that can irritate the eyes and the skin surrounding the eyes. Examples of traditional pH modifiers include basic pH modifiers, such as ammonia, mono-, di- and tri-alkyl amines, mono-, di- and tri-alkanolamines, alkali metal and alkaline earth metal hydroxides, and acidic pH modifiers, such as mineral acids and polycarboxylic acids. Therefore, surfactants can be employed to regulate the pH of the composition in the absence of traditional modifiers. Both foaming and non-foaming surfactants, including anionic, nonionic, and amphoteric surfactants, can be suitable pH altering surfactants. In one embodiment, cocoamphodiacetate disodium can be used as the pH modifying surfactant.

In one embodiment, the medicated eyelid scrub composition is configured for admixture with or mixed with an eyelid cleansing composition to form a medicated eyelid cleansing composition. An exemplary eyelid cleansing composition has been described in Applicant's U.S. Pat. No. 7,951,387, the contents of which are incorporated by reference herein.

In an exemplary first embodiment, the medicated eyelid scrub composition can include azithromycin dihydrate, about 0.5%-5% w/w of 0.9% sodium chloride solution, phenylethyl alcohol and polysorbate 80. According to an embodiment, the medicated eyelid scrub composition includes about 0.1%-2.0% w/w azithromycin dihydrate, about 0.5%-5% w/w of 0.9% sodium chloride solution, about 0.001%-0.05% w/w phenylethyl alcohol and 1%-5% polysorbate 80. In a specific embodiment, the medicated eyelid scrub composition includes about 1% w/w azithromycin dihydrate, about 0.02% w/w phenyl ethyl alcohol, about 2% w/w polysorbate 80, and about 0.5%-5% w/w of 0.9% sodium chloride solution.

In an exemplary second embodiment, the medicated eyelid scrub composition can include azithromycin dihydrate, diclofenac sodium, about 0.5%-5% w/w of 0.9% sodium chloride solution, phenyl ethyl alcohol and polysorbate 80. In one exemplary embodiment, the medicated eyelid scrub composition includes about 0.1%-2.0% w/w azithromycin dihydrate, about 0.05%-4.0% w/w diclofenac sodium, about 0.5%-5% w/w of 0.9% sodium chloride solution, about 0.001%-0.05% w/w phenylethyl alcohol and 1%-5% polysorbate 80. In one specific embodiment, the medicated eyelid scrub composition includes about 1% w/w azithromycin dihydrate, about 0.1% w/w diclofenac sodium, about 0.01% w/w phenyl ethyl alcohol, about 2% w/w polysorbate 80, and about 0.5%-5% w/w of 0.9% sodium chloride solution.

These medicated eyelid scrub compositions are mixed with a first eyelid cleansing composition that comprises Cocoamphodiacetate Disodium, D-Panthenol, Decyl Polygluoside, Methyl Gluceth-20, Modified Ringer's Solution (purified water, sodium chloride, potassium chloride, calcium chloride), PEG-120 Methyl Glucose Dioleate, POE-80 Sorbitan Monolaurate, Polyaminopropyl Biguanide, 1,2-Hexanediol and 1,2-Octanediol. In one or more embodiments, the API phase, that is, the medicated eyelid scrub composition, can be around 6%-10% w/w while the first eyelid cleansing composition can be around 90%-94% w/w of the formulation. However, it is understood that these amounts will vary with different percentages of medicated scrub composition.

In an embodiment, a method of preparing the medicated eyelid scrub composition involves mixing azithromycin dihydrate alone or with diclofenac sodium with polysorbate 80, phenyl ethyl alcohol and 0.5%-5% w/w of 0.9% sodium chloride solution at a temperature from 25° C. to 50° C. The resulting solution is then mixed with the first eyelid cleansing composition. The final solution is pH adjusted using hydrochloric acid or sodium hydroxide solutions to the pH range of 6 to 7.5.

Azithromycin, a macrolide antibiotic known for its broad-spectrum antibacterial activity. Azithromycin is particularly effective against Staphylococcus aureus, Staphylococcus epidermidis, and other bacterial pathogens commonly implicated in blepharitis and meibomian gland dysfunction. By incorporating Azithromycin into the medicated eyelid scrub composition, not only debris and biofilm are removed, but it also facilitates delivery of localized antimicrobial therapy to reduce bacterial load and prevent recurrent infections. Azithromycin inhibits bacterial protein synthesis, reducing pathogen proliferation and biofilm formation, a key contributor to chronic blepharitis.

Diclofenac is a nonsteroidal anti-inflammatory drug (NSAID). It can block cyclooxygenase (COX) enzymes, leading to reduced prostaglandin synthesis and alleviation of inflammation, redness, and discomfort. Beneficially, while azithromycin addresses bacterial overgrowth, the incorporation of diclofenac in the medicated eyelid scrub composition provides potent anti-inflammatory and analgesic effects, reducing eyelid swelling, irritation, and discomfort. This dual-action approach is particularly beneficial for patients suffering from chronic blepharitis, ocular rosacea, meibomian gland dysfunction, and allergic eyelid irritation.

In an exemplary third embodiment, the medicated eyelid scrub composition can include azithromycin dihydrate, dexamethasone phosphate, polysorbate 80, phenyl ethyl alcohol and sodium chloride solution. In an exemplary embodiment, the medicated eyelid scrub composition can include about 0.1%-2.0% w/w azithromycin dihydrate, about 0.05%-4.0% w/w dexamethasone phosphate, about 0.5%-5% w/w of 0.9% sodium chloride solution or injection, about 0.001%-0.05% w/w phenylethyl alcohol; and about 1%-5% w/w polysorbate 80. In one specific embodiment, the medicated eyelid scrub composition includes about 1% azithromycin dihydrate 1%, about 0.1% w/w dexamethasone phosphate, about 0.02% w/w phenyl ethyl alcohol, about 2% w/w polysorbate 80, and about 5% w/w of 0.9% sodium chloride solution.

Dexamethasone is known to inhibit the production of inflammatory mediators. The combination of azithromycin with dexamethasone, a glucocorticoid corticosteroid with potent anti-inflammatory properties, in the medicated eyelid scrub composition, is beneficial for treatment of bacterial overgrowth along with inflammatory conditions such as post-surgical swelling, allergic conjunctivitis to reduce inflammation, redness, and discomfort.

The medicated eyelid scrub composition is mixed with a first eyelid cleansing composition comprising cocoamphodiacetate disodium, D-Panthenol, decyl polygluoside, methyl Gluceth-20, modified Ringer's Solution (purified water, sodium chloride, potassium chloride, calcium chloride), PEG-120 Methyl Glucose Dioleate, POE-80 Sorbitan Monolaurate, polyaminopropyl biguanide, 1,2-Hexanediol and 1,2-Octanediol. In one or more embodiments, the API phase, that is, the medicated eyelid scrub composition, can be around 6%-10% w/w while the first eyelid cleansing composition can be around 90%-94% w/w of the formulation. However, it is understood that these amounts will vary with different percentages of the medicated scrub composition.

In an embodiment, the method of preparing medicated eyelid scrub composition involves mixing azithromycin dihydrate and dexamethasone phosphate with polysorbate 80, phenyl ethyl alcohol and 0.9% sodium chloride solution at a temperature from 25° C. to 50° C. The resulting solution is then mixed with the first eyelid cleansing composition. The final solution is pH adjusted using hydrochloric acid or sodium hydroxide solutions to the pH range of 6 to 7.5.

In an exemplary fourth embodiment, the medicated eyelid scrub composition includes metronidazole and ivermectin incorporated into an ointment base for treatment of demodex mites and ocular rosacea. In one exemplary embodiment, the medicated eyelid scrub composition includes about 0.01%-2% w/w metronidazole, and about 0.1%-2% w/w ivermectin. Metronidazole is an antibiotic used to treat skin infections and ocular rosacea and ivermectin is an antiparasitic used in treating demodex mites. Metronidazole inhibits organism protein synthesis by interacting with DNA causing organism cell death while ivermectin enhances the permeability of parasites causing paralysis and death by binding to glutamate-gated chloride channels. In a specific embodiment, the medicated eyelid scrub composition includes about 0.5% w/w metronidazole and about 1% w/w ivermectin.

The medicated eyelid scrub composition is mixed with about 20% to 60% of a suitable ointment base, such as, a 20% to 60% of a plasticized anhydrous ointment base consisting of mineral oil and polyethylene and a second eyelid cleansing composition comprising cocoamphodiacetate disodium, D-Panthenol, Decyl Glucoside, Methyl Gluceth-20, Modified Ringer's Solution (purified water, sodium chloride, potassium chloride, calcium chloride), PEG-120 Methyl Glucose Dioleate, PEG-80, Sorbitan Monolaurate, polyaminopropyl biguanide, 1,2-Hexanediol, Caprylyl Glycol, Polysorbate 20, Melaleuca Alternifolia (Tea Tree) Leaf Oil, Cocos Nucifera (Coconut) Oil. In one or more embodiments, the API phase/medicated eyelid scrub can be around 6%-10% w/w while the second eyelid cleansing composition can be around 20%-50% w/w and the ointment phase can be around 20%-50% w/w of the formulation. However, it is understood that these amounts will vary with different percentages of the medicated eyelid scrub composition.

In an embodiment, the method of preparing medicated eyelid scrub composition involves mixing metronidazole and ivermectin with the second eyelid cleansing composition. The resulting solution is pH adjusted using hydrochloric acid or sodium hydroxide solutions to the pH range of 6 to 7.5 and then mixed with a suitable ointment base.

In an embodiment, the medicated eyelid cleansing composition may be foaming or non-foaming. The medicated eyelid cleansing composition can be in liquid, cream, lotion, paste, spray, mist, gel, foam or ointment form.

An embodiment of the invention involves using the medicated eyelid scrub composition on a substrate that can cleanse eyelids. The substrate is capable of holding a therapeutically effective amount of the composition and can take the form of a wipe, a towelette, a fabric pad, or any other absorbent material. The term “pad” refers to a thick fabric that can retain the composition, and the fabric can be made of materials like rayon or cotton. Optionally, the fabric pad may have a textured surface.

The one or more embodiments of the medicated eyelid scrub compositions disclosed herein provide a non-invasive, easy-to-use therapeutic option for managing bacterial and inflammatory eyelid conditions, reducing the need for systemic antibiotics or steroid-based treatments. Patients who use the formulations comprising the medicated eyelid scrubs can experience improved symptom relief, including decreased crusting, redness, swelling, and discomfort, with regular use. The topical application ensures localized efficacy while minimizing systemic absorption, reducing potential side effects associated with oral antibiotics and NSAIDs.

Thus, the combination of the one or more medicated eyelid scrub compositions disclosed herein admixed with an eyelid cleansing composition offers a significant advancement in eyelid hygiene and treatment, providing healthcare providers with an effective, multi-functional solution for managing bacterial infections and inflammatory eyelid conditions. Beneficially, the compositions deter bacterial growth, inhibit biofilm formation, and alleviate swelling and discomfort. Their unique formulation allows for both cleansing and treatment in a single step, enhancing patient compliance and therapeutic outcomes. The compositions are therefore ideal for the targeted treatment of chronic blepharitis, ocular rosacea, and allergic irritation as part of a daily maintenance routine to prevent recurrent infection and inflammation.

Ávila et al. evaluated the efficacy of a combinatorial therapy using ivermectin and metronidazole for treating Demodex-related blepharitis (Ávila et al., Cont. Lens Anterior Eye, 2021). The study enrolled sixty patients, administering topical ivermectin (0.1%) and metronidazole (1%) to the treatment group at three time points over thirty days. Compared to the control group, which received only the topical vehicle, the treatment led to a significant elimination of Demodex mites (>96%). Additionally, the authors observed a notable reduction in inflammation and related symptoms in the treatment group, with no significant adverse effects directly attributed to the therapy. However, they acknowledged that the study lacked a comparison to single-agent treatments and an objective assessment of patient-reported symptoms.

Applicant conducted experimental surveys on patients that received the medicated lid scrub disclosed herein, and in particular, the one or more embodiments involving azithromycin, azithromycin and diclofenac, and azithromycin and dexamethasone. About 83% of the survey respondents believed it to be effective in treating their condition. Additionally, 91% expressed a willingness to use it again, indicating a high level of satisfaction with the treatment. The prescribing provider confirmed, during a follow-up consultation after the use of the medicated lid scrub to manage bacterial and inflammatory eyelids, that patients experienced a noticeable reduction in redness and swelling. In addition, the eyelids appeared healthier, with improvements in overall skin appearance and condition. These positive changes indicated that the treatment had a beneficial effect, contributing to better eye health and comfort for the patients.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. A medicated eyelid cleansing composition, comprising:
(A) a medicated eyelid scrub composition, comprising:
(i) azithromycin;
(ii) sodium chloride solution;
(iii) phenyl ethyl alcohol; and
(iv) polysorbate 80; and
(B) a first eyelid cleansing composition.

2. The medicated eyelid cleansing composition according to claim 1, wherein the medicated eyelid scrub composition further comprises:
about 0.1%-2.0% w/w azithromycin dihydrate;
about 0.5%-5% w/w of 0.9% sodium chloride solution;
about 0.001%-0.05% w/w phenylethyl alcohol; and
about 1%-5% polysorbate 80.

3. The medicated eyelid cleansing composition according to claim 1, wherein the medicated eyelid scrub composition further comprises diclofenac sodium.

4. The medicated eyelid cleansing composition according to claim 3, wherein the medicated eyelid scrub composition further comprises:
about 0.1%-2.0% w/w azithromycin dihydrate;
about 0.05%-4.0% w/w diclofenac sodium;
about 0.5%-5% w/w of 0.9% sodium chloride solution;
about 0.001%-0.05% w/w phenylethyl alcohol; and
about 1%-5% polysorbate 80.

5. The medicated eyelid cleansing composition according to claim 1, wherein the medicated eyelid scrub composition further comprises dexamethasone phosphate.

6. The medicated eyelid cleansing composition according to claim 5, further comprises:
about 0.1%-2.0% w/w azithromycin dihydrate;
about 0.05%-4.0% w/w dexamethasone phosphate;
about 0.5%-5% w/w of 0.9% sodium chloride solution;
about 0.001%-0.05% w/w phenylethyl alcohol; and
about 1%-5% polysorbate 80.

7. The medicated eyelid cleansing composition according to claim 1, wherein the first eyelid cleansing composition comprises: Cocoamphodiacetate Disodium, D-Panthenol, Decyl Polygluoside, Methyl Gluceth-20, Modified Ringer's Solution, PEG-120 Methyl Glucose Dioleate, POE-80 Sorbitan Monolaurate, Polyaminopropyl Biguanide, 1,2-Hexanediol and 1,2-Octanediol.

8. The medicated eyelid cleansing composition according to claim 1, wherein the medicated eyelid scrub composition is around 6%-10% w/w and the first eyelid cleansing composition is around 90%-94% w/w of the medicated eyelid cleansing composition.

9. The medicated eyelid cleansing composition according to claim 1, wherein the pH of the medicated eyelid cleansing composition is around 6 to 7.5.

10. The medicated eyelid cleansing composition according to claim 1, being a foaming or non-foaming formulation.

11. The medicated eyelid cleansing composition according to claim 1, being in the form of a liquid, cream, lotion, paste, spray, mist, gel, foam or ointment.

12. A medicated eyelid cleansing composition, comprising:
(A) a medicated eyelid scrub composition, comprising:
(i) metronidazole; and
(ii) ivermectin; and
(B) a second eyelid cleansing composition.

13. The medicated eyelid cleansing composition according to claim 12, comprising about 0.01%-2% w/w metronidazole and about 0.1%-2% w/w ivermectin.

14. The medicated eyelid cleansing composition according to claim 12, further comprising an ointment base.

15. The medicated eyelid cleansing composition according to claim 14, wherein the ointment base is a plasticized anhydrous ointment base consisting of mineral oil and polyethylene.

16. The medicated eyelid cleansing composition according to claim 14, further comprising about 20% to 60% of the ointment base.

17. The medicated eyelid cleansing composition according to claim 12, wherein the second eyelid cleansing composition further comprises: cocoamphodiacetate disodium, D-Panthenol, Decyl Glucoside, Methyl Gluceth-20, Modified Ringer's Solution, PEG-120 Methyl Glucose Dioleate, PEG-80, Sorbitan Monolaurate, polyaminopropyl biguanide, 1,2-Hexanediol, Caprylyl Glycol, Polysorbate 20, Melaleuca Alternifolia (Tea Tree) Leaf Oil, Cocos Nucifera (Coconut) Oil.

18. The medicated eyelid cleansing composition according to claim 14, wherein the medicated eyelid scrub composition is around 6%-10% w/w, the second eyelid cleansing composition is around 20%-50% w/w, and the ointment base is around 20%-50% w/w of the medicated eyelid cleansing composition.

19. The medicated eyelid cleansing composition according to claim 12, wherein the pH of the medicated eyelid cleansing composition is around 6 to 7.5.

20. The medicated eyelid cleansing composition according to claim 12, being a foaming or non-foaming formulation.

21. The medicated eyelid cleansing composition according to claim 12, being in the form of a liquid, cream, lotion, paste, spray, mist, gel, foam or ointment.
